(19) United States
(12) Patent Application Publication
(10) Pub. No.: US 2022/0268749 A1
(43) Pub. Date: Aug. 25, 2022

(54) TRACKING AND AUDITING OF GHG EMISSIONS

(71) Applicant: Microsoft Technology Licensing, LLC, Redmond, WA (US)

(72) Inventors: Mehmet Kadri UMAY, Redmond, WA (US); Imran SIDDIQUE, Bellevue, WA (US); Hari Krishnan SRINIVASAN, Redmond, WA (US); Nayana Singh PATEL, Mercer Island, WA (US); Jyothsna Devi BIJJAM, Redmond, WA (US)

(73) Assignee: Microsoft Technology Licensing, LLC, Redmond, WA (US)

(21) Appl. No.: 17/314,762

(22) Filed: May 7, 2021

Related U.S. Application Data
(60) Provisional application No. 63/153,538, filed on Feb. 25, 2021.

(51) Int. Cl. G01N 33/00; G06Q 50/06

(52) U.S. Cl. CPC G01N 33/0075; G01N 33/0047; G01N 33/0063; G01N 33/0031; G06Q 50/06

(57) ABSTRACT

One example provides a computing system configured to implement a greenhouse gas (GHG) tracking and auditing platform for tracking GHG emissions associated with a plurality of GHG entities, each GHG entity representing one or more of a GHG source and a GHG sink. The computing system comprises a logic subsystem and a data-holding subsystem comprising computer-readable instructions. The instructions are executable to receive sensor data over time from each sensor of one or more sensors, each sensor configured to sense data related to GHG emissions. The instructions are further executable to, for each time interval of a plurality of time intervals, determine a GHG emission amount based at least in part on the sensor data and store GHG emission data. The instructions are further executable to receive a request for a set of GHG emission data and in response to the request, output the set of GHG emission data.

### CROSS REFERENCE TO RELATED APPLICATIONS

This application claims priority to U.S. Provisional Patent Application Ser. No. 63/153,538, titled TRACKING AND AUDITING OF GHG EMISSIONS and filed Feb. 25, 2021, the entirety of which is hereby incorporated herein by reference for all purposes.

### BACKGROUND

The energy industry is rapidly moving to reduce greenhouse gas (GHG) emissions and transition to a GHG neutral future. Likewise, other industries, such as agriculture, forestry, and GHG capture initiatives, are growing as offsetting technologies.

### SUMMARY

Examples are disclosed that relate to tracking and auditing GHG emissions associated with each GHG entity of a plurality of GHG entities via a GHG tracking and auditing platform, each GHG entity representing one or more of a GHG source and a GHG sink. One example provides a computing system comprising a logic subsystem comprising one or more logic computational devices and a data-holding subsystem comprising computer-readable instructions executable by the logic subsystem. The instructions are executable to receive, for each GHG entity, sensor data over time from each sensor of one or more sensors, each sensor configured to sense data related to GHG emissions by the GHG entity and based at least in part on the sensor data, determine a GHG emission amount for each time interval of a plurality of time intervals for the GHG entity. The instructions are further executable for each time interval, store GHG emission data comprising the GHG emission amount determined, the time interval, and the sensor data. The instructions are further executable to receive a request for a set of GHG emission data for the entity and in response to the request, output the set of GHG emission data.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

### BRIEF DESCRIPTION OF THE DRAWINGS

FIG. 1 shows a block diagram of a use environment including an example greenhouse gas (GHG) tracking and auditing platform.

FIG. 2 shows example GHG data stored in the GHG tracking and auditing platform of FIG. 1.

FIGS. 3A and 3B show an example method for tracking and auditing GHG emission via the GHG tracking and auditing platform.

FIG. 4 shows an example computing system.

### DETAILED DESCRIPTION

As mentioned above, the energy industry is increasingly implementing measures to track GHG emissions in an effort to reduce GHG emissions worldwide. Such emissions include carbon dioxide (CO2), methane (CH4) and other hydrofluorocarbons (HFCs), perfluorocarbons (PFCs), sulfur hexafluoride (SF6), and nitrogen trifluoride (NF3), as examples.

Currently, GHG emissions are reported to various regulators annually, and report generation is commonly done though manual processes that may be inefficient and prone to error. Further, various different regulators may have different regulatory reporting standards, further complicating the manual processing and calculating of GHG emissions.

Accordingly, examples are disclosed that relate to a greenhouse gas (GHG) tracking and auditing platform that may provide solutions to such problems as those described above. Briefly the disclosed examples ingest GHG emissions data from sensors (real and virtual), compute GHG emissions based upon the ingested data, and store data related to GHG emissions in a manner that may allow different entities (e.g. energy companies, carbon sequestration/mitigation companies, manufacturing companies, and/or regulatory authorities) to access the data on demand and in a common format. Further, the platform may provide processes to generate audit reports that comply with the various regulatory standards, and/or allow trading of carbon credits between various entities. The term “emissions” may be used herein to represent both positive emissions and negative emissions (sequestration).

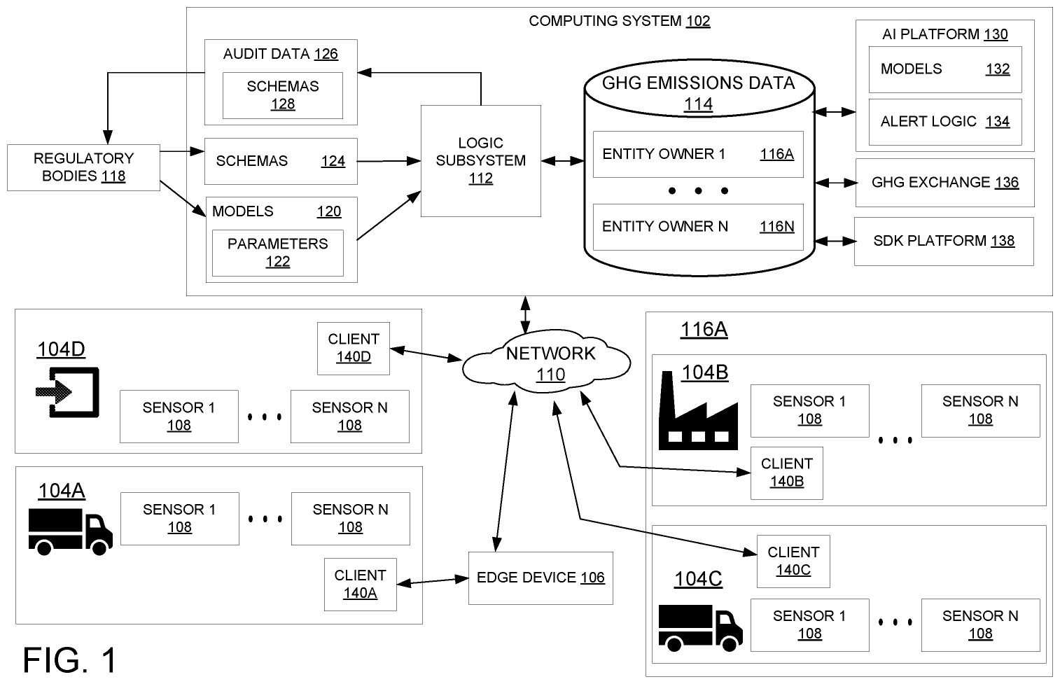

FIG. 1 shows a block diagram of an example use environment for tracking GHG emissions. The use environment comprises a computing system 102 implementing a greenhouse gas (GHG) tracking and auditing platform. Computing system 102 may be implemented as a cloud computing service, wherein the term “cloud computing service” represents a range of computing services, including compute power and data storage (illustrated as Infrastructure as a Service (IaaS) and Platform as a Service (PaaS)), delivered on-demand via the internet. Computing system 102 is in communication with one or more sources of sensor data for each of a plurality of GHG entities 104 via a network 102, such as the internet. As described in more detail below, computing system 102 is configured to receive sensor data (raw and/or processed) from sensors associated with GHG entities to track net emissions and/or sequestrations of GHG by each monitored entity. Each GHG entity 104 represents one or more of a GHG sink or a GHG source. A GHG source is a source of GHG emissions released into the atmosphere, whereas a GHG sink removes GHG from the atmosphere. As an example, GHG entity 104A is a transportation entity, here depicted as a truck, and thus is a source of GHG emissions. Other examples of transportation entities include trains, ships, and aircraft. GHG entity 104B is an energy production entity, such as a power plant that operates on natural gas or coal. While such an entity may be a net GHG source, the entity also may include a GHG capture infrastructure configured to capture GHG emissions and prevent the GHG emissions from being released into the atmosphere, and thus may have characteristics of a source and a sink. In such an example, the capture infrastructure may be considered a separate entity from the GHG-producing components of the power plant.

Multiple entities can be associated with an entity owner. Here, GHG entity 104B is owned by an owner 116A, such as a power company, that also owns transportation entities 104C. As such, GHG emissions data for multiple entities can be associated with the entity owner 116A for auditing and tracking carbon credits or debits. As another example entity, GHG entity 104D represents a GHG sequestration entity that removes GHG from the atmosphere on a net basis. GHG entity 104D may earn GHG credits that can be traded with GHG-emitting entity owners. GHG credits may be represented by tokens, as discussed in more detail below. It will be understood that these examples of GHG entities are presented for example and are not intended to be limiting, as computing system 102 may receive data related to any suitable entity related to GHG emissions.

Each GHG entity 104 comprises one or more sensors 108 each configured to sense data related to GHG emissions. Each sensor 108 can be a physical sensor, a virtual sensor, or any suitable combination therein. A physical sensor directly senses GHG emissions while a virtual sensor measures GHG emissions, e.g. by using data not directly linked to GHG emissions but that can be used to calculate GHG emissions based upon the sensor data. Each GHG entity 104 further comprises a client application 140 configured to interface with the computing system 102. Client application 140 can facilitate providing sensor data, receiving of alerts and/or control instructions, and performing other suitable communication between the GHG entity 104 and the computing system 102. In some instances, an entity may communicate sensor data to an edge device 106, which can then process the data and upload processed data (as well as raw data in some scenarios, such as when an anomalous event is detected by the edge device) to the computing system 102. Any suitable sensors may be used. In other examples, sensors can directly upload raw or locally processed data. Examples include, but are not limited to, temperature sensors, chemical sensors (e.g. gas sensors), flow rate sensors, pressure sensors, motion sensors, location sensors, image sensors, light sensors, and sound sensors. In various scenarios, these and other sensors can provide information on GHG emissions by entities.

As mentioned above, computing system 102 is configured to receive raw and/or processed sensor data, and store GHG emission amounts for each entity over time. AI platform 130 may host AI models that can be used to analyze GHG emissions data received from sensors related to entities, for example, to find probable anomalies in emissions by the entity. In some instances, logic subsystem 112 may apply one or more of models 120 as part of a GHG data ingestion process.

Logic subsystem 112 is further configured to store GHG data in a data-holding subsystem, and retrieve GHG emissions data 114 for various purposes, such as for analysis of emissions, auditing of emissions and/or trading of GHG credits. GHG emissions data 114 comprises data for one or more entity owners 116 (e.g. entity owner 116A), each entity owner 116 representing data associated with each GHG entity 104. Further details of the GHG emissions data 114 will be explained in more detail below. In the figures herein, the use of an “N” label (e.g. entity owner N and sensor N) indicates an arbitrary number of the labeled item, and does not mean that N has a same value for different labeled items.

Computing system 102 can also facilitate interactions with regulatory bodies 118, such as government agencies (e.g. the United States Environment Protection Agency), and/or any other agency that tracks and/or regulates GHG emissions. In some examples, computing system 102 can provide capabilities for secure data storage and secure communication between computing system 102 and regulatory bodies 118 and/or GHG entities 104, and/or can otherwise facilitate reporting of GHG emissions data to regulatory bodies. For example, computing system 102 may be configured to receive an audit request, search GHG emissions data 114 based upon the audit request, and output audit data 126 based at least in part on the GHG emissions data 114 retrieved from the data-holding subsystem in response to the audit request. Audit data 126 may be requested by an entity owner and/or by a regulatory body 118, as examples.

Further, to facilitate responding to audit requests, computing system 102 may store one or more data schemas 128 representing mappings of GHG data to disclosure standards. Such data schemas can be provided by regulatory bodies 118 in some examples. Audit data 126 can be organized via a selected data schema 128 to generate regulatory reports for submission to a requesting regulatory body 118. Thus, the use of data schemas to automatically format data for regulatory reporting may reduce the effort to generate the regulatory reports compared to current, more manual methods. Audit data 126 may include any suitable data for responding to audit requests. For example, audit data 126 can contain GHG emissions numbers as determined from sensor data, raw sensor data, and computations used to derive GHG emissions numbers from sensor data, to provide a transparent lineage of the GHG emissions data presented. In addition, logic subsystem 112 can translate an audit report for a different regulatory body by organizing audit data 126 to a schema for the different regulatory body.

As mentioned previously, computing system 102 can host an artificial intelligence (AI) platform 130. The AI platform 130 may run one or more models 132 to process GHG emissions data and identify patterns and deviations therefrom, as examples. Such models may include one or more neural networks, regression models, decision trees, support vector machines, K-nearest neighbors, random decision forests, and/or any other suitable type of model, depending upon the type of data being analyzed. As a more specific example, a model 132 can be configured as a leak detection model that analyzes sensor data related to the transportation or storage of GHG materials (e.g. methane) by an entity, where such a model can be trained based upon sensor data associated with prior known leak incidents.

As mentioned above, the AI platform further may comprise or otherwise interface with alert logic 134. Alert logic 134 can be configured, for example, to output an alert when an AI model predicts or detects an anomalous event from analyzing sensor data. The alert can comprise sensor data associated with the anomalous event detected. As a more specific example, an AI model may be configured to output a probability that sensor data represents a leak incident, and the alert logic 134 may be triggered to output an alert when the probability exceeds a threshold probability. The use of AI may allow the use of sensor data that preceded the leak incident to predict the risk of a future leak, and thereby allow preemptive action to be taken. A GHG entity 104 may be configured to take an action when the alert from the alert logic 134 is received. In the leak incident example, certain process may be automatically shut down or otherwise put in a safe mode to possibly reduce the impact of the leak incident. Further, alert logic 134 also may be triggered based upon raw and/or processed sensor data itself, in addition to or alternatively to a probability output by an AI model. While described in terms of leak detection, alert logic may output alerts based upon any other suitable types of events. As another example, an alert may be automatically generated when computing system 102 determines that a GHG entity 104 is nearing a target emission amount for a month or other suitable time frame. The GHG entity 104 may automatically change operations to reduce GHG emissions in order to not pass the target emission amount, or may automatically include the alert in an operational summary.

In some examples, computing system 102 further comprises a GHG exchange 136. GHG exchange 136 may be used to allow different entity owners to buy or sell GHG emissions credits (which may be referred to as GHG tokens), based upon the GHG emissions data tracked by computing system 102. Such GHG credits may be determined based at least partially on a GHG target and the GHG emission amount determined.

In some examples, computing system 102 can also host a software development kit (SDK) platform 138. The SKD platform 138 may enable a GHG entity to develop a specific application for a particular handling of GHG emissions data 114, such as generating an automated status report for example. It will be understood that any suitable application or application program interface for handling, auditing, reporting, or analyzing the GHG emissions data is also envisioned as being implemented via the SDK platform 138. Further, computing system 102 can provide connectivity in the form of providing a standard interface from GHG emissions data 114 to external applications implemented via other computing platforms as an example. The external applications can be data repositories or exchanges that can be standard, public, or private in nature.

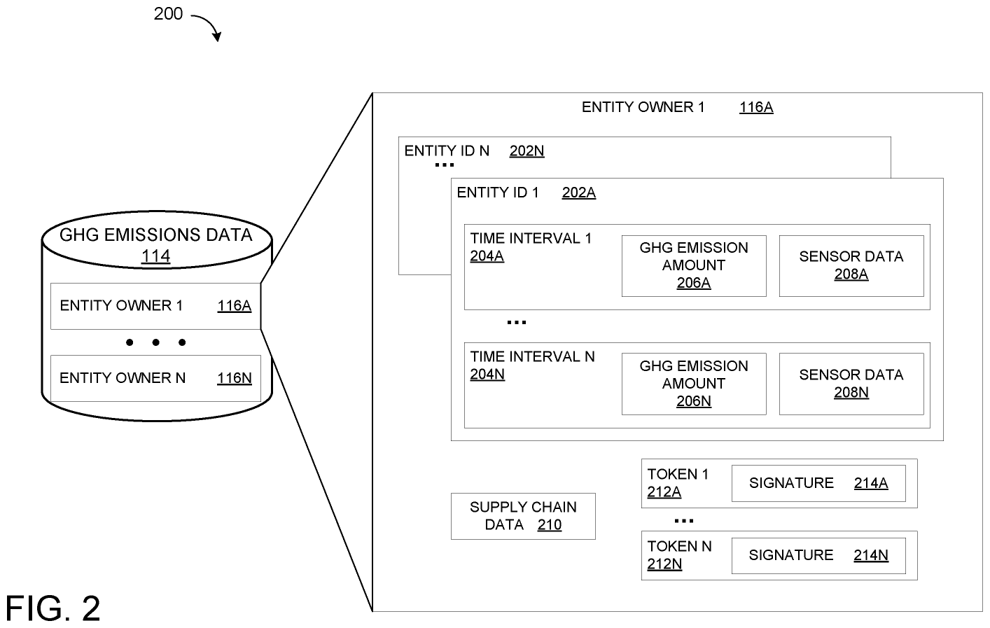

GHG data 114 may take any suitable form. FIG. 2 illustrates a schematic depiction of example GHG emissions data 114. GHG emissions data 114 comprises emissions-related data for each of one or more entity owners 116, wherein each entity owner 116 is associated with one or more GHG entities 104. In this example, entity owner 116A comprises an arbitrary number N of associated GHG entities, each identified by an entity identification (ID) 202, wherein each entity ID represents a different GHG source or GHG sink. For each time interval 204 of a plurality of time intervals, logic subsystem 112 stores the time interval 204, the GHG emission amount determined 206 and the sensor data 208 (raw and/or processed) in GHG emissions data 114. Other information may be stored as well, such as identifications of one or more models used to derive GHG emissions data. FIG. 2 illustrates two entity owners 116, two entity ID 202, and two time intervals 204, but any suitable number N of entity owners 116, entity ID 202, and time intervals 204 can be stored in GHG emissions data 114.

In other examples, entity owner 116 data may further comprise supply chain data 210 that represents the identities of entity owners in the supply chain of entity owner 116 and GHG emissions of such entity owners. In an energy production entity example, an oil refinery source can have supply chain data 210 related to crude oil that is being refined. As a non-limiting example, supply chain data 210 can comprise GHG emissions related to the production and transportation of the crude oil before reaching the oil refinery. Entity owner 116 can further comprise one or more tokens 212. The one or more tokens 212 can be bought or sold on an GHG exchange, such as GHG exchange 136. Each token 212 may represent a GHG credit generated by a GHG sink or other GHG-related activity for which credits are given. For security, in some examples each token 212 may comprises a signature 214 by a signing authority responsible for ensuring the authenticity of GHG credit data. For example, the signature may represent that a regulatory authority has reviewed GHG data related to the GHG sink responsible for generating the GHG credit, as well as the entity owner.

As mentioned previously, GHG emissions data 114 can be implemented via a data-holding subsystem which can comprise one or more database structures. Other suitable data-holding structures are also envisioned and can be implemented via a cloud system or a web server system as examples.

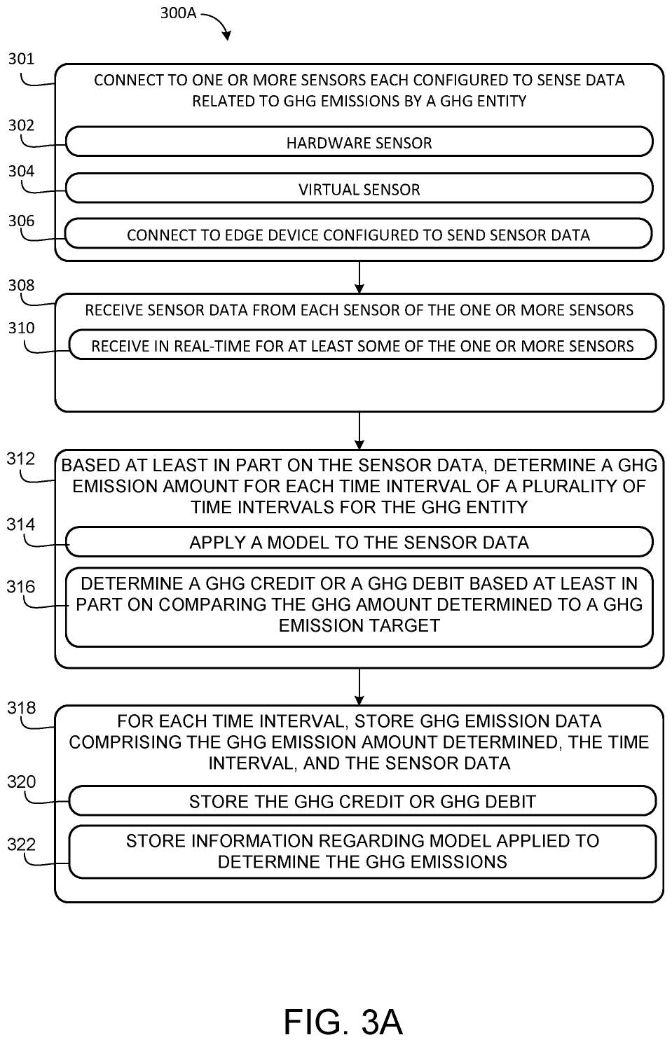

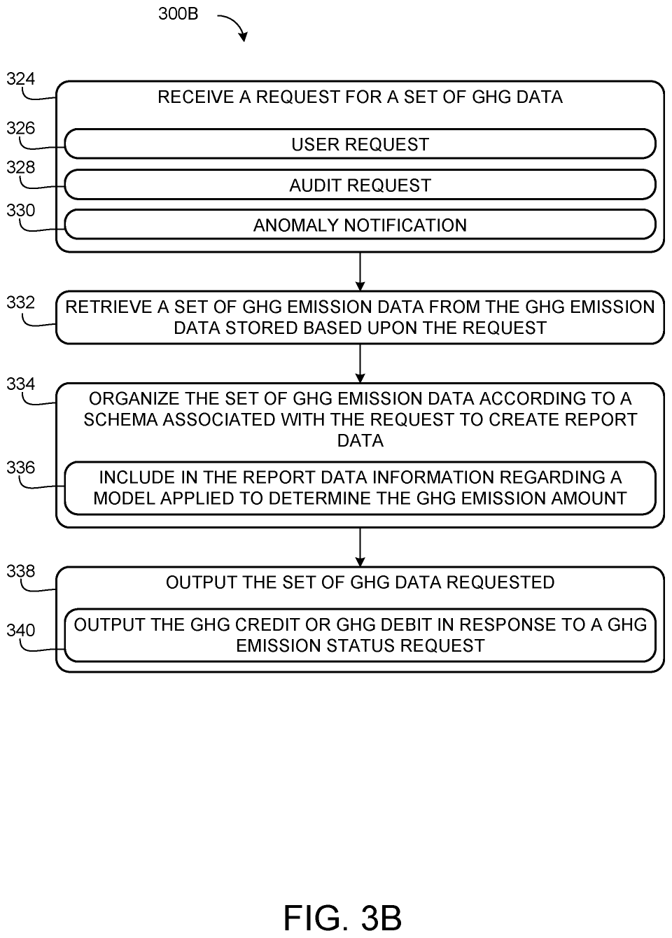

FIG. 3A and FIG. 3B illustrate a flow diagram depicting an example method 300 for tracking and auditing GHG emission data. Method 300 may be performed via any suitable computing system, such as cloud platform comprising computing devices located at one or more data centers (e.g. a plurality of geographically dispersed data centers). The cloud platform can further comprise edge devices configured to provide a subset of functionalities of the cloud platform at a location local to a GHG entity owner's network. In one example, method 300 may be implemented via the computing system 102 illustrated in FIG. 1. Method 300 comprises, at 301, connect to one or more sensors each configured to sense data related to GHG emissions by a GHG entity. Each GHG entity represent one or more of a GHG source and/or a GHG sink, and can be an industrial processing entity, a transportation entity, an energy production entity, or a GHG sequestration entity, as examples. The one or more sensors can be any suitable combination of one or more of hardware sensors 302 and/or virtual sensors 304, where hardware sensors are configured to directly sense the GHG emissions, while virtual sensors are configured to indirectly sense the GHG emissions and provide an output that can be converted to a GHG emissions value via application of a model. In some examples, method 300 can connect to an edge device configured to receive, process and/or send sensor data, at 306. The edge device may be configured to facilitate data flow between a network and the one or more sensors, for example, by sending processed sensor data instead of raw sensor data to reduce bandwidth usage.

Method 300 further comprises, at 308, receiving sensor data from each sensor of the one or more sensors. The sensor data is received over time, whether as a stream in real-time or near real time, or in batches, and is associated with time intervals during which the sensor data was collected. At 310, method 300 comprises receive the sensor data in real-time for at least some of the one or more sensors, in some examples. The sensor data can be received in real-time or near real-time as part of an internet-of-things solution or other suitable connectivity model.

Method 300 comprises, at 312, based at least in part on the sensor data, determine a GHG emission amount for each time interval of a plurality of time intervals for the GHG entity. In some examples, method 300 further comprises, at 314, determine a GHG emission amount based at least in part on applying a model to the sensor data. In some examples, the GHG emissions sensed indirectly by the virtual sensor can have the model applied to determine the GHG emission amount. In some examples, the model can be provided and maintained by a regulatory body. Further, in some examples, method 300 further comprises determine a GHG credit or a GHG debit based at least in part on comparing the GHG amount determined to a GHG emission target, at 316. The GHG credit or the GHG debit can be traded among GHG entities for example.

At 318, method 300 further comprises, for each time interval, storing GHG emission data comprising the GHG emission amount determined, the time interval, and the sensor data. In examples where a GHG credit or the GHG debit was determined, method 300 further comprises storing the GHG credit or GHG debit, at 320. In some examples, method 300 may additionally comprise, at 322, storing information regarding the model applied to determine the GHG emissions. The storing of both sensor data and model information used to determine a GHG emission amount may provide a transparent lineage of the GHG emission amount determination that can be retrieved at a later point in time.

At 324, method 300 comprises receive a request for a set of GHG data. The request can be a user request 326, an audit request 328, and/or an anomaly notification 330, in some examples. Method 300 further comprises, at 332, retrieving a set of GHG emission data from the GHG emission data stored based upon the request. In some examples, at 334, method 300 may comprise organizing the set of GHG emission data according to a schema associated with the request to create report data. In some such examples, the schema can be provided and maintained by a regulatory body and can be used to present the report data to a specified regulatory standard. Further, in some examples, when the GHG emission amount was determined in part by applying the model, method 300 comprises, at 336, include in the report data information regarding the model applied to determine the GHG emission amount. At 338, method 300 further comprises output the set of GHG data requested. When the GHG credit or GHG debit determined was stored, method 300 comprises, at 340, output the GHG credit or GHG debit in response to an GHG emission status request.

In some embodiments, the methods and processes described herein may be tied to a computing system of one or more computing devices. In particular, such methods and processes may be implemented as a computer-application program or service, an application-programming interface (API), a library, and/or other computer-program product.

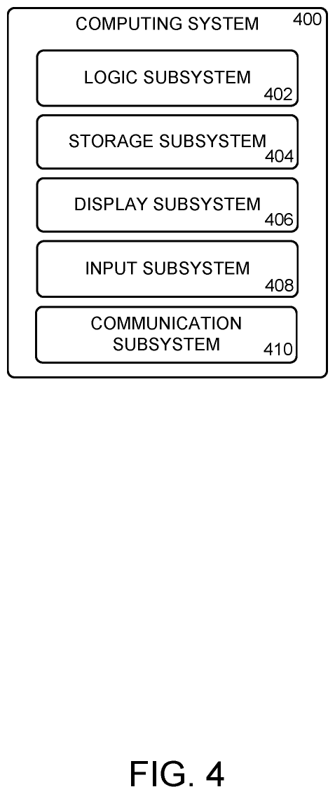

FIG. 4 schematically shows a non-limiting embodiment of a computing system 400 that can enact one or more of the methods and processes described above. Computing system 400 is shown in simplified form. Computing system 400 may embody the computing system 102, network 110 and/or edge device 106 in FIG. 1 and the GHG emissions data 114 described above and illustrated in FIG. 2. Further, example method 300 may be implemented via example computing system 400. Computing system 400 may take the form of one or more personal computers, server computers, tablet computers, home-entertainment computers, network computing devices, mobile computing devices, mobile communication devices (e.g., smart phone), and/or other computing devices, and wearable computing devices such as smart wristwatches and head mounted augmented reality devices.

Computing system 400 includes a logic subsystem 402 and a storage subsystem 404. Storage subsystem 404 can be any suitable combination there in of a volatile memory storage device and/or a non-volatile memory storage device. Computing system 400 may optionally include a display subsystem 406, input subsystem 408, communication subsystem 410, and/or other components not shown in FIG. 4.

Logic subsystem 402 includes one or more physical devices configured to execute instructions. For example, the logic subsystem may be configured to execute instructions that are part of one or more applications, programs, routines, libraries, objects, components, data structures, or other logical constructs. Such instructions may be implemented to perform a task, implement a data type, transform the state of one or more components, achieve a technical effect, or otherwise arrive at a desired result.

The logic subsystem may include one or more physical processors (hardware) configured to execute software instructions. Additionally or alternatively, the logic subsystem may include one or more hardware logic circuits or firmware devices configured to execute hardware-implemented logic or firmware instructions. Processors of the logic subsystem 402 may be single-core or multi-core, and the instructions executed thereon may be configured for sequential, parallel, and/or distributed processing. Individual components of the logic subsystem optionally may be distributed among two or more separate devices, which may be remotely located and/or configured for coordinated processing. Aspects of the logic system may be virtualized and executed by remotely accessible, networked computing devices configured in a cloud-computing configuration. In such a case, these virtualized aspects are run on different physical logic processors of various different machines, it will be understood.

Storage subsystem 404 may include one or more physical devices configured to hold instructions executable by the logic processors to implement the methods and processes described herein. When such methods and processes are implemented, the state of storage subsystem 404 may be transformed—e.g., to hold different data.

Storage subsystem 404 may include physical devices that are removable and/or built-in. Storage subsystem 404 may include optical memory (e.g., CD, DVD, HD-DVD, Blu-Ray Disc, etc.), semiconductor memory (e.g., ROM, EPROM, EEPROM, FLASH memory, etc.), and/or magnetic memory (e.g., hard-disk drive, floppy-disk drive, tape drive, MRAM, etc.), or other mass storage device technology. Storage subsystem 404 may include volatile, nonvolatile, dynamic, static, read/write, read-only, sequential-access, location-addressable, file-addressable, and/or content-addressable devices.

Aspects of logic subsystem 402, storage subsystem 404 may be integrated together into one or more hardware-logic components. Such hardware-logic components may include field-programmable gate arrays (FPGAs), program- and application-specific integrated circuits (PASIC/ASICs), program- and application-specific standard products (PSSP/ASSPs), system-on-a-chip (SOC), and complex programmable logic devices (CPLDs), for example.

When included, display subsystem 406 may be used to present a visual representation of data held by storage subsystem 404. The visual representation may take the form of a graphical user interface (GUI). As the herein described methods and processes change the data held by the non-volatile storage device, and thus transform the state of the storage subsystem 404, the state of display subsystem 406 may likewise be transformed to visually represent changes in the underlying data. Display subsystem 406 may include one or more display devices utilizing virtually any type of technology. Such display devices may be combined with logic subsystem 402, storage subsystem 404 in a shared enclosure, or such display devices may be peripheral display devices.

When included, input subsystem 408 may comprise or interface with one or more user-input devices such as a keyboard, mouse, touch screen, or game controller. In some embodiments, the input subsystem may comprise or interface with selected natural user input (NUI) componentry. Such componentry may be integrated or peripheral, and the transduction and/or processing of input actions may be handled on- or off-board. Example NUI componentry may include a microphone for speech and/or voice recognition; an infrared, color, stereoscopic, and/or depth camera for machine vision and/or gesture recognition; a head tracker, eye tracker, accelerometer, and/or gyroscope for motion detection and/or intent recognition; as well as electric-field sensing componentry for assessing brain activity; and/or any other suitable sensor.

When included, communication subsystem 410 may be configured to communicatively couple various computing devices described herein with each other, and with other devices. Communication subsystem 410 may include wired and/or wireless communication devices compatible with one or more different communication protocols. As non-limiting examples, the communication subsystem may be configured for communication via a wireless telephone network, or a wired or wireless local- or wide-area network, such as a HDMI over Wi-Fi connection. In some embodiments, the communication subsystem may allow computing system 400 to send and/or receive messages to and/or from other devices via a network such as the Internet.

Another example provides a computing system configured to implement a greenhouse gas (GHG) tracking and auditing platform for tracking GHG emissions associated with each GHG entity of a plurality of GHG entities, each GHG entity representing one or more of a GHG source and a GHG sink. The computing system comprises a logic subsystem comprising one or more logic computational devices; and a data-holding subsystem comprising computer-readable instructions executable by the logic subsystem to, for each GHG entity, receive sensor data over time from each sensor of one or more sensors, each sensor configured to sense data related to GHG emissions by the GHG entity; based at least in part on the sensor data, determine a GHG emission amount for each time interval of a plurality of time intervals for the GHG entity; for each time interval, store GHG emission data comprising the GHG emission amount determined, the time interval, and the sensor data; receive a request for a set of GHG emission data for the entity; and in response to the request, output the set of GHG emission data. In some examples, the plurality of GHG entities comprise one or more of an industrial processing entity, a transportation entity, an energy production entity, or a GHG sequestration entity. In some examples, the instructions are alternatively or additionally executable to determine the GHG emission amount for one or more of the plurality of GHG entities based at least in part on applying a model to the sensor data. In some examples, the instructions executable to store the GHG emission amount are alternatively or additionally executable to store information regarding the model applied. In some examples, the request comprises an audit request. In some examples, the instructions executable to receive sensor data over time are alternatively or additionally executable to receive sensor data over time in real-time for at least some of the one or more sensors. In some examples, the instructions are alternatively or additionally executable to detect an anomalous GHG emission amount, and output an alert indication that the anomalous GHG emission amount was detected.

Another example provides a method enacted on a computing system configured to implement a greenhouse gas (GHG) tracking and auditing platform for tracking GHG emissions associated with each entity of a plurality of GHG entities, each GHG entity representing one or more of a GHG source and a GHG sink. The method comprises, for each GHG entity, receiving sensor data over time from each sensor of the one or more sensors, each sensor configured to sense data related to GHG emissions by the GHG entity; based at least in part on the sensor data, determining a GHG emission amount for each time interval of a plurality of time intervals for the GHG entity; for each time interval, storing GHG emission data comprising the GHG emission amount determined, the time interval, and the sensor data; receiving a request for a set of GHG emission data for the entity; and in response to the request, outputting the set of GHG emission data. In some examples, the plurality of GHG entities comprise one or more of an industrial processing entity, a transportation entity, an energy production entity, or a GHG sequestration entity. In some examples, the method alternatively or additionally comprises determining the GHG emission amount for one or more of the plurality of GHG entities based at least in part on applying a model to the sensor data. In some examples, storing the GHG emission amount alternatively or additionally comprises storing information regarding the model applied. In some examples, the request alternatively or additionally comprises an audit request. In some examples, receiving sensor data over time alternatively or additionally comprises receiving sensor data over time in real-time for at least some of the one or more sensors. In some examples, the method alternatively or additionally comprises detect an anomalous GHG emission amount, and output an alert indication that the anomalous GHG emission amount was detected.

Another example provides a method for reporting GHG emissions associated with each entity of a plurality of GHG entities, each GHG entity representing one or more of a GHG source and a GHG sink. The method comprises receiving GHG emission data for one or more of the GHG entities, the GHG emission data comprising a GHG emission amount determined, a time interval, and sensor data; storing the GHG emission data; receiving a request for a report; retrieving a set of GHG emission data from the GHG emission data stored based upon the request; organizing the set of GHG emission data according to a schema associated with the report to create report data; and outputting the report data. In some examples, the method alternatively or additionally includes in the report data information regarding a model applied to determine the GHG emission amount. In some examples, receiving the GHG emission data for one or more of the GHG entities alternatively or additionally comprises receiving GHG emission data over time in real-time for at least some of the one or more of the GHG entities. In some examples, the plurality of GHG entities comprises one or more of an industrial processing entity, a transportation entity, an energy production entity, or carbon sequestration entity. In some examples, the method alternatively or additionally comprises processing the set of GHG emission data to determine a GHG emission anomaly, and wherein retrieving the set of GHG emission data from the GHG emission data stored alternatively or additionally comprises retrieving GHG emission data from the GHG emission data stored related to the GHG emission anomaly. In some examples, the method alternatively or additionally comprises detecting an anomalous GHG emission amount, and outputting an alert indicating that the anomalous GHG emission amount was detected. In some examples, the method alternatively or additionally comprises determining a GHG credit or a GHG debit based at least in part on comparing the GHG amount determined to a GHG emission target; storing the GHG credit or GHG debit, and outputting the GHG credit or GHG debit in response to a request for information on the GHG credit or GHG debit.

Another example provides a computing system configured to implement a greenhouse gas (GHG) tracking and auditing platform for tracking GHG emissions associated with each GHG entity of a plurality of GHG entities, each GHG entity representing one or more of a GHG source and a GHG sink. The computing system comprising a logic subsystem comprising one or more logic computational devices, and a data-holding subsystem comprising computer-readable instructions executable by the logic subsystem. The instructions are executable to, for each GHG entity, receive sensor data over time from each sensor of one or more sensors, each sensor configured to sense data related to GHG emissions by the GHG entity, based at least in part on the sensor data, determine a GHG emission amount for each time interval of a plurality of time intervals for the GHG entity, for each time interval, store GHG emission data comprising the GHG emission amount determined, the time interval, and the sensor data, receive a request for a set of GHG emission data for the entity, and in response to the request, output the set of GHG emission data. In some examples, the plurality of GHG entities alternatively or additionally comprise one or more of an industrial processing entity, a transportation entity, an energy production entity, or a GHG sequestration entity. In some examples, the instructions are alternatively or additionally executable to determine the GHG emission amount for one or more of the plurality of GHG entities based at least in part on applying a model to the sensor data. In some examples, the instructions alternatively or additionally are executable to store the GHG emission amount are further executable to store information regarding the model applied. In some examples, the request alternately or additionally comprises an audit request. In some examples, the instructions alternatively or additionally are executable to receive sensor data over time are executable to receive sensor data over time in real-time for at least some of the one or more sensors. In some examples, the instructions are alternately or additionally executable to detect an anomalous GHG emission amount, and output an alert indication that the anomalous GHG emission amount was detected.

Another example provides a method enacted on a computing system configured to implement a greenhouse gas (GHG) tracking and auditing platform for tracking GHG emissions associated with each entity of a plurality of GHG entities, each GHG entity representing one or more of a GHG source and a GHG sink. The method comprises, for each GHG entity, receiving sensor data over time from each sensor of the one or more sensors, each sensor configured to sense data related to GHG emissions by the GHG entity, based at least in part on the sensor data, determining a GHG emission amount for each time interval of a plurality of time intervals for the GHG entity, for each time interval, storing GHG emission data comprising the GHG emission amount determined, the time interval, and the sensor data, receiving a request for a set of GHG emission data for the entity, and in response to the request, outputting the set of GHG emission data. In some examples, the plurality of GHG entities alternatively or additionally comprise one or more of an industrial processing entity, a transportation entity, an energy production entity, or a GHG sequestration entity. In some examples, the method alternatively or additionally comprises determining the GHG emission amount for one or more of the plurality of GHG entities based at least in part on applying a model to the sensor data. In some examples, storing the GHG emission amount alternately or additionally comprises information regarding the model applied. In some examples, receiving sensor data over time alternatively or additionally receiving sensor data over time in real-time for at least some of the one or more sensors. In some examples, the method alternatively or additionally comprises detecting an anomalous GHG emission amount, and output an alert indication that the anomalous GHG emission amount was detected.

Another example provides a method for reporting GHG emissions associated with each entity of a plurality of GHG entities, each GHG entity representing one or more of a GHG source and a GHG sink. The method comprising receiving GHG emission data for one or more of the GHG entities, the GHG emission data comprising a GHG emission amount determined, a time interval, and sensor data, storing the GHG emission data, receiving a request for a report, retrieving a set of GHG emission data from the GHG emission data stored based upon the request, organizing the set of GHG emission data according to a schema associated with the report to create report data, and outputting the report data. In some examples, the method alternatively or additionally comprises including in the report data information regarding a model applied to determine the GHG emission amount. In some examples, receiving the GHG emission data for one or more of the GHG entities alternately or additionally comprises receiving GHG emission data over time in real-time for at least some of the one or more of the GHG entities. In some examples, the plurality of GHG entities alternatively or additionally comprises one or more of an industrial processing entity, a transportation entity, an energy production entity, or carbon sequestration entity. In some examples, the method alternatively or additionally comprises processing the set of GHG emission data to determine a GHG emission anomaly, and wherein retrieving the set of GHG emission data from the GHG emission data stored further comprises retrieving GHG emission data from the GHG emission data stored related to the GHG emission anomaly. In some examples, the method alternatively or additionally comprises detecting an anomalous GHG emission amount, and outputting an alert indicating that the anomalous GHG emission amount was detected. In some examples, the method alternatively or additionally comprises determining a GHG credit or a GHG debit based at least in part on comparing the GHG amount determined to a GHG emission target, storing the GHG credit or GHG debit, and outputting the GHG credit or GHG debit in response to a request for information on the GHG credit or GHG debit.

It will be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated and/or described may be performed in the sequence illustrated and/or described, in other sequences, in parallel, or omitted. Likewise, the order of the above-described processes may be changed.

The subject matter of the present disclosure includes all novel and non-obvious combinations and sub-combinations of the various processes, systems and configurations, and other features, functions, acts, and/or properties disclosed herein, as well as any and all equivalents thereof.

1. A computing system configured to implement a greenhouse gas (GHG) tracking and auditing platform for tracking GHG emissions associated with each GHG entity of a plurality of GHG entities, each GHG entity representing one or more of a GHG source and a GHG sink, the computing system comprising:
a logic subsystem comprising one or more logic computational devices; and
a data-holding subsystem comprising computer-readable instructions executable by the logic subsystem to, for each GHG entity,
receive sensor data over time from each sensor of one or more sensors, each sensor configured to sense data related to GHG emissions by the GHG entity;
based at least in part on the sensor data, determine a GHG emission amount for each time interval of a plurality of time intervals for the GHG entity;
for each time interval, store GHG emission data comprising the GHG emission amount determined, the time interval, and the sensor data;
receive a request for a set of GHG emission data for the entity; and
in response to the request, output the set of GHG emission data.

2. The computing system of claim 1, wherein the plurality of GHG entities comprise one or more of an industrial processing entity, a transportation entity, an energy production entity, or a GHG sequestration entity.

3. The computing system of claim 1, wherein the instructions are further executable to determine the GHG emission amount for one or more of the plurality of GHG entities based at least in part on applying a model to the sensor data.

4. The computing system of claim 3, wherein the instructions executable to store the GHG emission amount are further executable to store information regarding the model applied.

5. The computing system of claim 1, wherein the request comprises an audit request.

6. The computing system of claim 1, wherein the instructions executable to receive sensor data over time are executable to receive sensor data over time in real-time for at least some of the one or more sensors.

7. The computing system of claim 1, wherein the instructions are further executable to detect an anomalous GHG emission amount, and output an alert indication that the anomalous GHG emission amount was detected.

8. A method enacted on a computing system configured to implement a greenhouse gas (GHG) tracking and auditing platform for tracking GHG emissions associated with each entity of a plurality of GHG entities, each GHG entity representing one or more of a GHG source and a GHG sink, the method comprising:
for each GHG entity,
receiving sensor data over time from each sensor of the one or more sensors, each sensor configured to sense data related to GHG emissions by the GHG entity;
based at least in part on the sensor data, determining a GHG emission amount for each time interval of a plurality of time intervals for the GHG entity;
for each time interval, storing GHG emission data comprising the GHG emission amount determined, the time interval, and the sensor data;
receiving a request for a set of GHG emission data for the entity; and
in response to the request, outputting the set of GHG emission data.

9. The method of claim 8, wherein the plurality of GHG entities comprise one or more of an industrial processing entity, a transportation entity, an energy production entity, or a GHG sequestration entity.

10. The method of claim 8, further comprising determine the GHG emission amount for one or more of the plurality of GHG entities based at least in part on applying a model to the sensor data.

11. The method of claim 10, wherein storing the GHG emission amount further comprises information regarding the model applied.

12. The method of claim 8, wherein receiving sensor data over time is receiving sensor data over time in real-time for at least some of the one or more sensors.

13. The method of claim 8, further comprising detect an anomalous GHG emission amount, and output an alert indication that the anomalous GHG emission amount was detected.

14. A method for reporting GHG emissions associated with each entity of a plurality of GHG entities, each GHG entity representing one or more of a GHG source and a GHG sink, the method comprising:
receiving GHG emission data for one or more of the GHG entities, the GHG emission data comprising a GHG emission amount determined, a time interval, and sensor data;
storing the GHG emission data;
receiving a request for a report;
retrieving a set of GHG emission data from the GHG emission data stored based upon the request;
organizing the set of GHG emission data according to a schema associated with the report to create report data; and
outputting the report data.

15. The method of claim 14, further comprising including in the report data information regarding a model applied to determine the GHG emission amount.

16. The method of claim 14, wherein receiving the GHG emission data for one or more of the GHG entities comprises receiving GHG emission data over time in real-time for at least some of the one or more of the GHG entities.

17. The method of claim 14, wherein the plurality of GHG entities comprises one or more of an industrial processing entity, a transportation entity, an energy production entity, or carbon sequestration entity.

18. The method of claim 17, further comprising
processing the set of GHG emission data to determine a GHG emission anomaly, and
wherein retrieving the set of GHG emission data from the GHG emission data stored further comprises retrieving GHG emission data from the GHG emission data stored related to the GHG emission anomaly.

19. The method of claim 14, further comprising detecting an anomalous GHG emission amount, and outputting an alert indicating that the anomalous GHG emission amount was detected.

20. The method of claim 14, further comprising:
determining a GHG credit or a GHG debit based at least in part on comparing the GHG amount determined to a GHG emission target;
storing the GHG credit or GHG debit; and
outputting the GHG credit or GHG debit in response to a request for information on the GHG credit or GHG debit.
